(19) United States
(12) Patent Application Publication
(10) Pub. No.: US 2010/0222423 A1
(43) Pub. Date: Sep. 2, 2010

(54) ASPALATHIN-LIKE DIHYDROCHALCONE, EXTRACTS FROM UNFERMENTED ROOIBOS AND PROCESS FOR PREPARATION

(75) Inventors: Bruno Frank, Kleinrinderfeld (DE); Wilfried Dimpfel, Wetzlar (DE)

Correspondence Address: SMITH PATENT CONSULTING, LLC, 515 East Braddock Road, Suite B, ALEXANDRIA, VA, 22314, US

(73) Assignee: KNEIPP-WERKE KNEIPP-MITTEL-ZENTRALE GMBH & CO. KG, Würzburg (DE)

(21) Appl. No.: 12/739,280

(22) PCT Filed: Sep. 5, 2008
(86) PCT No.: PCT/EP2008/007279

§ 371 Date: Apr. 29, 2010

(30) Foreign Application Priority Data
Oct. 23, 2007 (EP) 07020728.7

(51) Int. Cl. A61K 31/352; C07D 407/08; A61P 25/16; A61P 25/24; A61P 25/18; A61P 25/28

(52) U.S. Cl. 514/456; 549/399

(57) ABSTRACT

The present invention relates to a novel chemical compound of the formula I and to the pharmaceutically acceptable salts, derivatives and esters thereof. In addition, the invention also relates to a process for isolating the compound according to formula I from raw rooibos material. The present invention likewise relates to a rooibos extract which has a content of the compound according to formula I of at least 0.4% by weight. The invention furthermore relates to the use of the chemical compound of the formula I and of the pharmaceutically acceptable salts, derivatives and esters thereof and of the rooibos extract according to the invention as a medicament, in particular for treating neurological and psychiatric disorders of the central nervous system. The expression “pharmaceutically active” also includes those effects which lead to a subjective improvement in the mental state, in which case approval under pharmaceutical legislation need not be absolutely necessary. Compound of the formula I is shown below:

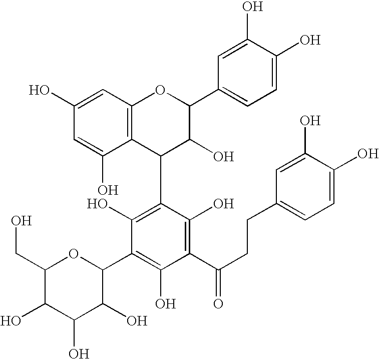

The present invention relates to a novel chemical compound of the formula I and to the pharmaceutically acceptable salts, derivatives and esters thereof. In addition, the invention also relates to a process for isolating the compound according to formula I from raw rooibos material. The present invention likewise relates to a rooibos extract which has a content of the compound according to formula I of at least 0.4% by weight, preferably at least 1.5% by weight. The invention furthermore relates to the use of the chemical compound of the formula I and of the pharmaceutically acceptable salts, derivatives and esters thereof and of the rooibos extract according to the invention as a medicament, in particular for preventing and treating neurological and psychiatric disorders of the central nervous system, in particular dementias. The expression “pharmaceutically active” also includes those effects which lead to a subjective improvement in the mental state, in which case approval under pharmaceutical legislation need not be absolutely necessary.

Rooibos (botanical name: Aspalathus linearis) grows exclusively in South Africa and is currently the only known plant worldwide which contains the particularly strongly antioxidant substance aspalathin, a flavonoide. In addition, rooibus contains further flavonoids, such as C-glycosylflavones (including orientin, isoorientin), flavonol-3-O-glycosides (including quercetin, quercitrin, isoquercitrin, rutin) and the dihydrochalcones nothofagin and aspalathin.

Unfermented “green” rooibos is distinguished by a higher content of polyphenols, in particular aspalathin, and a higher antioxidant activity in comparison with the fermented products. The effect of reducing the antioxidant activity by the fermentation process is equally observable in the case of black compared with green tea (Bramati et al., J. Agric. Food Chem. 2003, 51: 7472-7474). Scientific investigations have shown that the antioxidant activity of rooibos tea is mainly derived from the aspalathin content. In the investigation into the fermentation process of rooibos tea, it was found that the content of aspalathin and nothofagin decreases during the fermentation process (Schulz et al., Eur. Food Res. Technol. 2003, 216: 539-543). The lower antioxidant activity of fermented “red” rooibos tea compared with unfermented “green” rooibos tea can be explained thereby.

Owing to the abovementioned health-promoting flavonoids and its good flavour, rooibos tea is widely consumed. Further ingredients of rooibos tea are phenolic acids, essential oil, vitamin C and numerous minerals, in particular iron and fluoride.

In order to achieve as high an antioxidant activity as possible, a high content of aspalathin is necessary. In this context, DE 10 2005 004 438 discloses a rooibos extract which, in comparison with the customary aspalathin content of 1 to 3% by weight, and has an increased content of more than 5% by weight in combination with a low chlorophyll content of less than 0.4% by weight. According to DE 10 2005 004 438, the rooibos extract is obtained by extracting unfermented raw rooibos material with the use of a mixture of ethanol and water, an ethanol/water mixture in the ratio of 80 to 20 being used. Owing to the strongly antioxidant, antiirritant and antimicrobial action, the rooibos extract having a high aspalathin content is said to be used in particular for cosmetic application, for example as hair care agents, skincare agents or oral hygiene agents.

A further flavonoid which is present in rooibos tea is quercitin. This occurs with a content of about 11 mg/100 g of raw rooibos material and influences, for example, the histamine release in the human body, with the result that allergic symptoms can be alleviated. Quercitin was also capable of inhibiting the production of monoaminooxidase, which advantageously influences mild depressions and sleep disorders (Plantextrakt [Plant extract], the nature network, issue 3 of Sep. 11, 2005; Plantextrakt GmbH).

A starting point of the present invention was a search for active substances for treating diseases of the central nervous system, such as, for example, dementias, Parkinson's disease, depression and pain. These diseases are difficult to treat, and the medicaments used here, such as, for example tacrine, galantamine or nefopam, have a broad range of adverse effects.

An object of the invention is therefore to provide active substances, compositions and extracts from rooibos for the therapeutic treatment of these diseases. In particular, these active substances or compositions should have a low level of adverse effects.

This object is achieved by the subject of the patent claims.

The present invention relates to a compound of the formula I

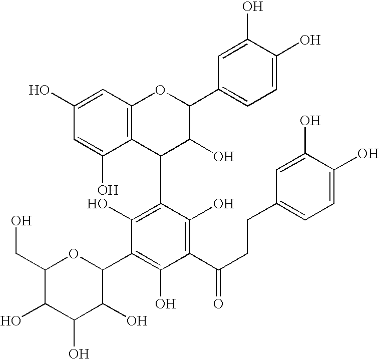

and to the pharmaceutically acceptable salts, derivatives and esters thereof. Preferred derivatives here are coupling products of the compound according to formula I with ferulic acid, quinic acid, caffeic acid, gluconic acid or chlorogenic acid. The compound of the formula I is preferably used in its natural form or as a salt, more preferably in its natural form according to formula I.

Among the preferred esters, formic acid, acetic acid, propionic acid, glutaric acid, tartaric acid or succinic acid esters may be mentioned. Preferred salts are the salts with cationic, organic or inorganic counterions, in particular alkali metal salts, alkaline earth metal salts, ammonium salts or salts with pharmaceutically acceptable acids, such as succinates, citrates, tartrates.

Furthermore, the invention relates to a rooibos extract, preferably from unfermented rooibos, having a content of the compound according to formula I of at least 0.4% by weight, more preferably at least 1.5% by weight, more preferably at least 2.5% by weight, more particularly preferably at least 5% by weight, even more preferably at least 10% by weight and most preferably at least 20% by weight.

Furthermore, the invention relates to a process for the preparation of the compound of the formula I, in which
dried and comminuted, unfermented raw rooibos material is extracted with an extracting agent consisting of methanol and/or water for a predetermined duration of extraction at a temperature of up to 90° C., preferably up to 60° C.,
the extract is filtered and then evaporated to dryness under reduced pressure, and then
purified by a plurality of chromatographic separation steps, preferably three chromatographic separation steps, with the use of two Sephadex LH20 columns and subsequently a lipophilic c18-HPLC column.

The invention also relates to the use of a rooibos extract as a medicament or food supplement. The invention moreover relates to the use of compounds according to formula I and of the pharmaceutically acceptable salts, derivatives and esters thereof as a medicament or food supplement. The food supplement or medicament may contain the rooibos extract, for example, in an amount of at least 10 mg, at least 20 mg or at least 50 mg per g of food supplement or per dosage unit of the medicament. Preferably, the food supplement or medicament contains the rooibos extract in an amount of at least 100 mg, more preferably of at least 200 mg, even more preferably of at least 300 mg, per g of food supplement or per dosage unit of the medicament. In a further preferred embodiment, the food supplement or medicament contains at least 1 mg, more preferably at least 2 mg, even more preferably at least 3 mg, even more preferably at least 5 mg and most preferably at least 10 mg of the compound of the formula I per g of food supplement or per dosage unit of a medicament.

In a preferred embodiment of the invention, the rooibos extract and compounds according to formula I and the pharmaceutically acceptable salts, derivatives and esters thereof are used with the increased proportion of the compound according to formula I as a drug for preventing or treating neurological or psychiatric disorders of the central nervous system, preferably for treating dementias, Parkinson's disease, depression and pain, in particular Alzheimer's disease.

In an even more preferred embodiment, the invention relates to the use of rooibos extract and of the compound of the formula I or of the pharmaceutically acceptable salts and esters thereof for the preparation of a medicament for preventing and/or treating neurological and psychiatric disorders of the central nervous system, wherein the neurological and psychiatric disorders of the central nervous system are dementias, Parkinson's disease, depression and pain, more preferably Alzheimer's disease.

Within the scope of the present invention, a novel, pharmacologically active natural substance could be isolated from a rooibos extract and then characterized. Unexpectedly, the novel chemical compound could be isolated only from unfermented “green” rooibos extract. Only complete characterization of the compound permitted the detection of very small amounts in the fermented rooibos extract too.

The process for isolating the novel chemical compound is described in detail in example 1, and the structural formula is shown in formula I. The compound of the formula I has similarities to both the structure of aspalathin and that of catechin(4α->2)phloroglucinol (FIG. 1). Exact characterization and the structure elucidation are described in detail in example 2. In comparison with the flavonoids known to date, the novel compound according to formula I has a high molecular weight of 740.66 g/mol. Such high molecular weight natural substances are not usually used for active substance screening since, owing to their molecular weight, they cannot easily penetrate the blood-brain barrier. Such substances therefore tend to be regarded as being unsuitable for the brain as a site of action and for the treatment of diseases of the central nervous system.

Unexpectedly, however, a bias-free investigation by Tele-Stereo-EEG (electroencephalography) of the rat showed a pronounced, central nervous, pharmacological activity of the compound according to the formula I. The pharmacological investigations surprisingly showed that the activity in the Tele-Stereo-EEG model in rats results in dose-dependent changes in the EEG frequencies, as are known following administration of classical medicaments for the treatment of dementias (for example galantamine or tacrine), Parkinson's disease (L-DOPA) and pain (for example nefopam).

Within the scope of the present invention, the pharmacological activity of the inventive compound according to formula I was compared with the known ingredients of rooibos extract, such as aspalathin, catechin or (−)-epicatechin. The experimental investigations are described in detail in example 3 and unexpectedly show that the action of the compound according to formula I cannot be achieved with approximately equimolar amounts of aspalathin, catechin or (−)-epicatechin, although the compound according to formula I has structural similarities with these natural substances. The novel inventive compound according to formula I has a higher pharmacological activity than these known ingredients of rooibos extract and is therefore particularly suitable for use as a medicament.

The isolation of this novel compound having advantageous pharmacological activities permits in particular the preparation of a medicament or food supplement based on the compound according to formula I, both as a monopreparation and in combination with further active substances. These further active substances can act in the same direction or have completely different properties advantageously influencing the clinical picture in another manner. The combination of this compound with other flavonoids present in rooibos and ingredients in the overall combination is also suitable for this purpose. In particular, ingredients which have an antioxidant action are suitable here.

A discriminant analysis of the in vivo data of the compound according to formula I showed, as mentioned above, a relationship to the medicaments for treating dementias, Parkinson's disease, depression and pain. Since these medicaments have a broad spectrum of adverse effects, the use of the compound according to formula I as a medicament is advantageous since natural substances are usually expected to have a lower frequency of adverse effects. Unexpectedly, the novel substance or the metabolites thereof moreover evidently cross the blood-brain barrier. This is generally not usual for flavonoids.

Furthermore, a preparation process for rooibos extracts and for the newly identified pharmacologically active compound is provided. The process according to the invention makes it possible to provide a particularly suitable plant extract for treating the abovementioned diseases.

The rooibos extract prepared according to the invention has a content of the compound according to formula I of at least 1% by weight, more preferably at least 1.5% by weight, even more preferably at least 2% by weight, more preferably at least 2.5% by weight, even more preferably at least 3% by weight and most preferably at least 5% by weight.

In a very particularly preferred embodiment, a highly concentrated rooibos extract is used. A highly concentrated rooibos extract comprises at least 10% by weight and preferably at least 20% by weight of the compound of the formula I. In a particular embodiment, the highly concentrated rooibos extract comprises at least 50% by weight of the compound of the formula I.

A process according to the invention for the preparation of the compound according to formula I comprises the following steps (also see example 1):
provision of dried and comminuted, unfermented raw rooibos material,
extraction of the provided raw material with an extracting agent consisting of a mixture of an alcohol, preferably methanol, and/or water for a predetermined duration of extraction at a temperature of up to 90° C., preferably up to 60° C.,
filtration of the extract,
evaporation of the filtered extract at a reduced pressure,
purification of the extract in three steps:
   rough purification by chromatography on a Sephadex LH20 column
   fine purification by chromatography on a further Sephadex LH20 column
   separation on a lipophilic c18-HPLC column.

In a particularly preferred embodiment, ascorbic acid is added in an amount of 0.001 to 1% by weight, preferably 0.01 to 0.2% by weight, in the extraction step of the process according to the invention. The percentages by weight are based on the weight of the drug to be extracted, i.e. plant parts, such as dried leaves or stems.

If preparations having a high content of compounds according to formula I are desired, the extract can also be separated using only one chromatography column.

Preferably, the moisture content of the raw rooibos material provided is 4% or less, since in this way autofermentation of the starting material is prevented.

For achieving a high yield of the compound of the formula I, an alcohol/water mixture in the ratio 50:50 to 80:20, depending on the alcohol used (methanol, ethanol, propanol, propan-2-ol are suitable), is used as an extracting agent according to a preferred configuration of the process according to the invention. Preferably, a 50:50 methanol/water mixture is used. In this preferred embodiment of the process according to the invention, the ratio of raw material to extracting agent is preferably about 1:6, and the extraction step is preferably effected at elevated temperature (above 40° C.) of 1 hour but is also possible at room temperature and with a duration of, for example, 2 to 5 hours.

The evaporation of the filtered extract is preferably effected at a pressure of less than 300 mbar. The temperature in the evaporation step is preferably not more than 40° C.

The rooibos extract according to the invention is prepared by the process described above, the rooibos extract being obtained after evaporation to dryness (without subsequent chromatographic purification).

The method of measurement for determining the content of the compound according to formula I in the rooibos extract is described in detail in example 4.

The compound according to formula I, the pharmaceutically acceptable salts, derivatives and esters thereof and the rooibos extract according to the invention are suitable in particular for treating diseases of the central nervous system, preferably dementias, Parkinson's disease, depression and pain and as a cell protection antioxidant or “free radical scavenger”. The treatment of dementias, such as senile dementia or Alzheimer's disease, is particularly preferred.

According to the invention, the novel compounds can be used as an individual active substance or in combination with further active substances, in the form according to formula I or as pharmaceutically acceptable salts or complexes and esters or derivatives. Suitable derivatives, salts, complexes and esters and the preparation thereof are known to the person skilled in the art. The preparation of pharmaceutically acceptable salts (hydrochloride, succinates, citrate, tartrate, etc) is likewise known to the person skilled in the art. Suitable salt formers are all customary pharmaceutically acceptable acids or anions. Furthermore, coupling to acids, such as, for example, ferulic acid, quinic acid, caffeic acid, gluconic acid, chlorogenic acid and related compounds is possible. In particular, coupling to gluconic acid is preferred. The coupling products of the compound according to formula I with the above acids are designated as pharmaceutically tolerated derivatives. However, the compound is preferably used as a molecule according to formula I.

In another, likewise preferred embodiment, the extracts are prepared by using a slightly more hydrophilic solvent mixture. This solvent mixture comprises at least one alcohol, preferably selected from methanol, ethanol, n-propanol or 2-propanol, and water. The alcohol content is between 10 and 50% by weight of alcohol. If this solvent mixture is chosen, the total proportion of flavonoid is increased in comparison with a solvent mixture in which higher proportions of alcohol are used. In this embodiment, the alcohol content is between 10 and 60% (vol/vol), preferably between 10 and 50% (vol/vol). Even more preferably, the alcohol content is between 15 and 40% and very particularly preferably between 20 and 30% (vol/vol).

In this embodiment of the process, the starting drug used is “green” rooibos having as low a residual moisture content as possible, which is preferably less than 5% (water per total weight). The starting material, namely leaves and sprigs of Aspalathus linearis (unfermented), is dried particularly rapidly and under mild conditions and then extracted with an alcohol-water mixture, the water content being >60% (vol/vol). In particularly preferred embodiments, the alcohol content is 15 to 25%, very particularly preferably 20%, of methanol or 25% to 35%, particularly preferably 30%, of ethanol.

In a very particularly preferred embodiment, pure alcohol is first added to the dried starting drug and, after a softening phase which usually lasts for between 30 and 60 minutes, the corresponding amount of water is added. After the extraction, the extract is filtered and the filtrate is evaporated to dryness under reduced pressure. This is preferably followed by a chromatographic purification step, the purification preferably being effected by use of a Sephadex column chromatography.

In this embodiment, compounds of the formula I are virtually completely extracted, at the same time a high proportion of the total flavonoids being extracted. The extracts (before a further purification by chromatography methods) have a content of compounds in the formula I which is between 2.5 and 5% (weight based on dry extract). The total flavonoid content, as the sum of the aspalathin types, of the rutoside types and vitexin types without a compound according to formula I, is between 15 and 30% by weight, based on the dry extract. The ratio of vitexin types (═C-glycosides) to rutoside types is 1,6. The content of aspalathin types is 14 to 25%, based on the weight of the dry extract.

In this method of carrying out the extraction process, the total flavonoid content of the extract is increased. Usually, such an extract (without additional purification by column chromatography) has at least about 20% by weight of the total flavonoids. The following main components could be identified in the extract:
a) compound of the formula I;
b) group of substances consisting of the chalcones (aspalathin and nothofagin);
c) rutoside group; this includes flavonoids which contain quercitin as a constituent, flavonoids which contain a sugar via a C—O—C linkage (quercitin is the aglycone of rurtin) and
d) the vitexin group; this is understood as meaning flavonoids which contain sugar via a C—C link; vitexin is the aglycone of apigenin, e.g. vitexin (apigenin-8-C-glucoside), isovitexin (apigenin-6-C-glucoside), orientin (luteolin-8-C-glucoside), isoorientin (lutoelin-6-C-glucoside).

Characteristic of this rooibos extract is, inter alia, the weight ratio of the individual groups to one another. Thus, the ratio of the group consisting of the vitexins to the rutoside group, based on the proportions by weight, is from 1:2 to 1:50, preferably between 1:3 and 1:10.

The extract according to the invention differs from those extracts for internal use which were obtained in the case of tea beverages, i.e. in the case of purely aqueous extraction, through higher contents of the compounds mentioned. The extracts according to the invention differ from extracts which were intended for external use, for example in cosmetics, by having a different ratio of the flavonoid groups to one another. Extracts which are intended for external use are obtained by extraction with a higher ethanol content (at least about 80% of ethanol). The extract obtained with 80% of ethanol was optimized for as high an aspalathin content as possible. The ratio of the three flavonoid groups (aspalathin-like, rutoside-like and vitexin-like ═C-glycosides) is changed by the relatively lipophilic extraction compared with the starting state in the drug. This is particularly clearly evident from the ratio of the vitexin-like to the rutoside-like flavonoids.

The rooibos extract according to the invention and the inventive compound according to formula I or the salts, derivatives and esters thereof, preferably the naturally occurring compound according to formula I, can be processed in a manner known per se to give medicaments and/or food supplements having health-promoting properties. The rooibos extract according to the invention and the inventive compound according to formula I and the salts, derivatives and esters thereof can be formulated, for example, in the form of tablets, capsules, pills, coated tablets, granules, powders, lozenges and liquid administration forms, such as, for example, drinks. Use in food supplements, in particular for hot and cold beverages, or as soluble tea is also preferred.

Preferably, the food supplement and medicament are administered orally, topical, parenteral, intravenous, intramuscular, subcutaneous, nasal, inhalative, rectal or transdermal application also being possible.

The rooibos extract according to the invention and the medicament or food supplement according to the invention may additionally preferably contain further active substances which enhance the effect of the rooibos extract or of the inventive compound according to formula I or the salts thereof or have a supplementary positive influence on the systems or conditions occurring in the case of said diseases (e.g.: further free radical scavengers, various enzyme-inhibiting substances, vitamins, lecithins, omega-3-fatty acids and substances which positively influence brain functions).

Furthermore, pharmaceutically acceptable excipients and carriers can be used. Suitable excipients are known to the person skilled in the art and comprise, for example, fillers, disintegrants, lubricants, binders, wetting agents, etc.

Suitable lubricants are, for example, silicate, talc, stearic acid, magnesium or calcium stearate and/or polyethylene glycols.

Binders which may be used are, for example, starches, gum arabic, gelatin, methylcellulose, carboxymethylcellulose or polyvinylpyrrolidone.

Disintegrants are, for example, starch, alginic acid, alginates or sodium starch glycolates or foaming mixtures.

Wetting agents which may be used are, for example, lecithin, polysorbates or laurylsulfates.

Furthermore, colorants and sweeteners may also be present in the formulations.

The pharmaceutical preparations can be prepared in a known manner, for example by means of mixing, granulation, tabletting or sugar-coating or overcoating methods.

The liquid dispersions and/or solutions for oral administration may be, for example, drinks, drops, syrups, emulsions and suspensions.

The syrup may contain, for example, sucrose or sucrose with glycerol and/or mannitol and/or sorbitol as a carrier.

The suspensions and the emulsions may contain, for example, a natural resin, agar, sodium alginate, pectin, methylcellulose, carboxymethylcellulose or polyvinyl alcohol as a carrier.

The suspensions or solutions for intramuscular injections may contain, together with the active substance, a pharmaceutically acceptable carrier, e.g. sterile water, olive oil, ethyl oleate, glycols, e.g. propylene glycol, and, if desired, a suitable amount of lidocaine hydrochloride.

The solutions for intravenous injection or infusion may contain, for example, sterile water as a carrier or they may preferably be present in the form of sterile, aqueous, isotonic salt solutions.

The suppositories may contain, together with the active substance, a pharmaceutically acceptable carrier, e.g. cocoa butter, polyethylene glycol, a polyoxyethylene sorbitol fatty acid ester or lecithin.

Compositions for topical application, e.g. creams, lotions or pastes, can be prepared by mixing the active substance with a conventional oil-containing or emulsifying carrier.

The rooibos extract according to the invention and the inventive compound according to formula I or the salts, derivatives and esters thereof can be used in customary amounts in the food supplements or medicaments. In solution, preferably 0.001 to 10% by weight of the inventive compound according to formula I or of the salts thereof, more preferably 0.1 to 7% by weight and particularly preferably 1 to 5% by weight, are used. In a particular embodiment, 0.02 to 1% by weight of the inventive compound according to formula I or of the salts thereof is used in solution.

The rooibos extract according to the invention is preferably used in amounts which correspond to an amount of the compound according to formula I of 1 to 1000 mg, more preferably 10 to 600 mg, even more preferably 50 to 400 mg and most preferably 50 to 250 mg.

If a rooibos extract which has a very high proportion of the compound according to formula I is used, such an extract can be employed in medicaments and in food supplements in an amount which is between 3 and 600 mg, preferably between 5 and 100 mg and particularly preferably between 10 and 50 mg per daily dose.

The food supplements or medicaments described above can be prepared by conventional methods and administered in a pharmaceutically suitable form.

The preferred, solid food supplements or medicaments according to the invention may additionally contain 1 to 95% by weight, preferably 1 to 50% by weight, more preferably 1 to 20% by weight, particularly preferably 1 to 10% by weight, of fillers.

Fillers which may be used are one or more compounds which provide a part of the material for achieving the required and desired tablet or capsule mass. Inter alia, microcrystalline cellulose in various particle sizes, in particular having a mean particle size in the range of from 20 μm to 200 μm, in particular in the range from 50 μm to 150 μm, such as, for example, about 100 μm, such as the known Avicel products, such as Avicel PH-101 and PH-102, can be used. Further suitable fillers are, for example, maize starch, potato starch, lactose, cellactose (a mixture of cellulose and lactose), calcium phosphate, dextrose, mannitol, maltodextrin, isomalt, silicone dioxide (Aerosil), optionally also sorbitol and sucrose. If direct compression is intended, it should be ensured when choosing the fillers that qualities are used which are suitable for the direct compression of tablets. In the case of the commercial products, this will be stated in each case by the manufacturer or can be checked by simple experiments. The most preferred filler is microcrystalline cellulose (commercial products are, for example, Avicel, Vivapur and Emcocel).

Suitable disintegrants are known in the prior art. Disintegrants preferred according to the invention are, for example crospovidones (Kollidon CL) and starch or pregelatinized starch, in particular the commercial product “Starch 1500”. Further suitable starches are commercially available, for example, under the names Lycatab PGS, Prejel and Sepistab ST 200.

Furthermore, the known so-called “Super Disintegrants” can also be used, such as croscarmellose sodium (e.g. Ac-Di-Sol, etc) and carboxymethyl-starch sodium (e.g. Explotab, Primojel, etc). Starches such as Starch 1500 are particularly preferred.

The content of disintegrant is a rule 1 to 25% by weight, preferably 1 to 20% by weight, in particular 2 to 15% by weight. Suitable ranges for the content of disintegrant are also, for example, 2 to 5% by weight or 15 to 20% by weight, depending on the disintegrants, fillers and other additives used.

According to the invention, the composition may contain, as lubricant, one or more compounds which support the preparation and the processing of the tablet. Lubricants which may be used are, inter alia, stearic acid and derivatives thereof, such as calcium stearate, and in particular sodium stearylfumarate (which is commercially available, for example, under the name Pruv) and magnesium stearate, glyceryl mono-, di- and in particular tristearate, hydrogenated vegetable oil (e.g. Lubritab, Dynasan, Sterotext) or a polyethylene glycol (e.g. Lutrol, Carbowax).

The content of lubricant is as a rule 0.1 to 4% by weight, preferably 0.2 to 4% by weight.

Optionally, the pharmaceutical composition according to the invention may comprise one or more flow regulators. Suitable flow regulators are magnesium trisilicate, talc and in particular silicone dioxide (e.g. Aerosil). If the composition comprises a flow regulator, this is present as a rule in an amount of 0.5 to 5% by weight, preferably 1 to 4% by weight, in particular 2 to 3% by weight.

The pharmaceutical compositions according to the invention may also contain stabilizers for the active substance, such as ascorbic acid, citric acid, tartaric acid, lactic acid, etc, preferably ascorbic acid or citric acid. The content of stabilizer (if present) is as a rule in the range from 0.1 to 10% by weight, 0.5 to 10% by weight, preferably 1 to 3% by weight.

The pharmaceutical compositions according to the invention may contain further customary pharmaceutically tolerated additives and excipients, where they preferably contain no further excipients apart from the abovementioned ones (filler, disintegrant, lubricant and optionally flow regulator and stabilizer).

Some fillers, such as microcrystalline cellulose, may also serve as a binder. Moreover, fillers having a binder function therefore count as the fillers in the context of this application.

If the pharmaceutical composition according to the invention is present as a tablet, it may be film-coated with one or more coating materials. Coating materials which may be used are shellac and shellac mixtures, hypromellose (hydroxypropylmethylcellulose), polyvinyl alcohol, sodium carboxymethylcellulose and various methacrylicacidpolymers (Eudragits), hypromellose, Eudragits, shellac and shellac mixtures being preferred. The coating of the tablets is effected in a customary manner. In addition to the coating material, further customary constituents of tablet coatings, such as plasticizers, pigments, pore formers or suspension stabilizers, may be present in the coating, such as, for example, polyethylene glycol (PEG), talc or titanium dioxide and optionally also lactose.

The tablet weight is not particularly limited; tablets with 100 mg to 500 mg with the use of pure active substances and 250 mg to 1500 mg, 500 mg to 1500 mg, with the use of extracts and plant powders are usual. In capsules, amounts of 100 mg to 1000 mg are used.

The dosage unit of the medicament or food supplement may, for example, contain:
in the case of peroral pharmaceutical forms:
preferably 1 to 1000 mg, more preferably 40 to 800 mg, particularly preferably 150 to 500 mg, even more preferably 300 to 600 mg, of rooibos extract per daily dose. With the use of the compound according to formula I and the pharmaceutically acceptable salts, derivatives and esters thereof, 1/100 to 1/20 of the above amounts are used.
The daily dose can be administered daily, for example, in 1 to 3 single doses, preferably in two single doses. It is also possible to envisage that 1-10 single doses of rooibos extract containing the compound according to formula I will be administered daily.
in the case of parenteral pharmaceutical forms (for example intravenously, subcutaneously, intramuscularly):
preferably 3 to 60 mg, particularly preferably 10 to 30 mg, of active substance per daily dose.
The daily dose can be administered daily, for example, in 1 to 3 single doses, preferably in one single dose.
in the case of pharmaceutical forms for rectal application:
preferably 40 to 80 mg, particularly preferably 60 mg, of active substance according to formula I of the extract per daily dose.
The daily dose may be administered daily, for example in 1 to 3 single doses, preferably in one single dose.
in the case of pharmaceutical forms for application to the skin or mucus membranes (e.g. solutions, lotions, emulsions, ointments, etc):
preferably 40 to 80 mg of active substance, particularly preferably 60 mg of active substance, per single dose. If the content of compound according to formula I relates to the prepared solution, lotion, emulsion or ointment, the portion by weight, based on such ointment-like medicaments, is then between 0.05 and 20% by weight, preferably between 0.2 and 1% by weight, of compound of the formula I, based on the cream-like preparation.
The daily dose can be administered daily, for example, in 1 to 6 single doses, preferably in 1 to 3 single doses.
With the use of a pharmaceutically acceptable salt, the amount used must be adapted appropriately in a manner known to the person skilled in the art.

### DESCRIPTION OF THE FIGURES

FIG. 1 shows the structural formula of aspalathin (1) and catechin(4α->2)phloroglucinol (2).

FIG. 2 shows the numbering of the atoms in the compound according to formula I.

FIG. 3 shows the assignment of the NMR (nuclear magnetic resonance) signals to the atoms with the numbers according to FIG. 2. Explanation of the legend: ^{*)/**)/+)/++)/§)/§§)/#)##)}=values can be interchanged between one another, ^{A), B)}=sum of the integral or chemical shift of the group stated only once.

FIG. 4 shows structure fragments A-D of the compound according to formula I, characterized by means of NMR experiments.

FIG. 5 shows table 2 with ¹³C-NMR data of the compound according to formula I in comparison with aspalathin and catechin(4α->2)phloroglucinol. Owing to the lack of ¹³C-NMR literature data on aspalathin, the assignment of the signals for the aspalathin isolated by HWI ANALYTIK GMBH was made on the basis of date from: Ho et al., Phytochemistry 1980, 19, 476-477. The data of catechin(4α->2)phloroglucinol were taken from Matthews et al., J. Agric. Food. Chem. 1997, 45, 1195-1201. Explanation of the legend: ^{*)/**)/+)/++)/§)/§§)/#)##)}=values can be interchanged with one another.

FIG. 6 shows evidence of stable experimental conditions on administration of a salt solution to Fischer-344 rats after administration of the compound according to formula I. The time in hours after application is plotted along the y axis. The x axis shows the frequency ranges after the fast Fourier Transformation of the data: delta, theta, alpha1, alpha2, beta1 and beta2.

FIG. 7 shows the effect of 3 mg/kg of the compound according to formula I on EEG frequencies up to 5 hours after application. Mean values of n=6 animals. Asterisks characterize the statistical significance, calculated according to Ahrens and Läuter, 1974. *=p<0.01; **=p<0.05; ***=p<0.025.

FIG. 8 shows the effect of 6 mg/kg of the compound according to formula I on EEG frequencies up to 5 hours after application. Mean values of n=6 animals. Asterisks characterize the statistical significance, calculated according to Ahrens and Läuter, 1974. *=p<0.01; **=p<0.05; ***=p<0.025.

FIG. 9 shows the effect of the compound of the formula I in comparison with its molecular constituents aspalathin, catechin or epicatechin. With approximately equimolar administration, the effect due to the unique structure can be achieved only by the compound according to formula I but not by parts of the molecule.

FIG. 10 shows the effect of 6 mg/kg of the compound of the formula I on EEG frequencies during the first hour after application. Mean values of n=6 animals. The similarity to medicaments which are used for treating dementia, Parkinson's disease, depression and pain should be noted: L-DOPA (Parkinson's disease); tacrine and galantamine (Alzheimer's disease); nefopam (pain treatment).

FIG. 11 shows the result of the motility measurement, which was carried out simultaneously with the measurement of the field potentials. Data are given in % of the starting value before administration of the substance. Only (−)-epicatechin causes a substantial increase in the motility.

FIG. 12 shows a UV spectrum of the compound having the formula I (G110907SA).

FIG. 13 shows a UV spectrum of aspalathin.

FIG. 14 shows a UV spectrum of rutoside.

FIG. 15 shows a UV spectrum of orientin.

FIG. 16 shows a UV spectrum of homoorientin.

FIG. 17 shows a UV spectrum of vitexin.

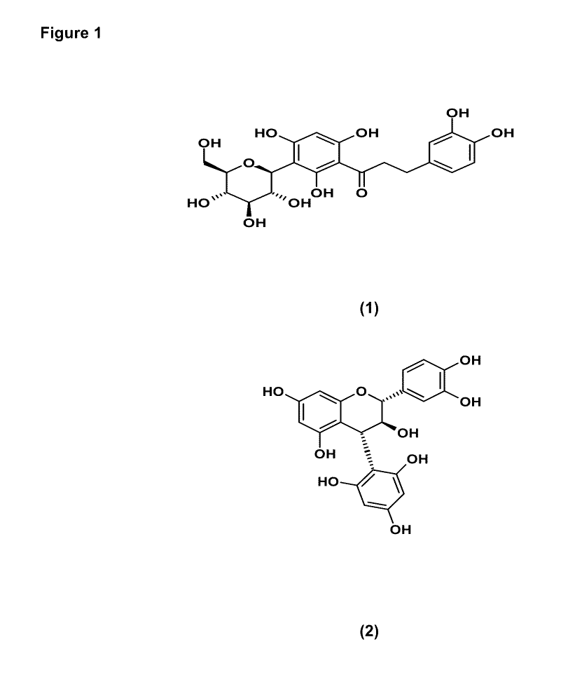

The present invention is explained in more detail by the following examples.

### EXAMPLE 1

### Isolation of the Compound According to Formula I

For the preparation of the extracts from the drug, unfermented green rooibos from the manufacturer Rooibos Ltd., Clanwilliam, South Africa, is used.

Unfermented and comminuted leaves and/or sprigs of Aspalathus linearis dried under mild conditions to a moisture content of less than 10% (preferably less than 4%) are used as starting material.

This raw material is extracted by means of a mixture of methanol and water in the ratio 50:50 (parts by volume) at 60° C. for 1 hour with rotation, the raw material:solvent ratio being 1:7. Thereafter, the liquid is filtered off from the plant parts and the plant parts are extracted again in the same manner and filtered.

The two filtrates are combined and are freed from methanol at reduced pressure (220 mbar) and 55° C. The remaining aqueous solution is diluted with water to 5 times the weight of the amount of dried plant parts used and is subjected to a liquid-liquid partition.

3 litres of the aqueous solution are shaken 4 times with 1.5 l of water-saturated n-butanol each time, and the combined butanol phases are brought to dryness under reduced pressure. The yield is about 10% of the amount of dried plant parts used.

This is followed first by a coarse separation and then by a fine separation of the butanol extract.

### Coarse Separation:

About 50 g of butanol extract are chromatographed over a Sephadex LH20 column (6 cm internal diameter and 80 cm height of fill=2260 ml Sephadex LH20) with 50% by volume of methanol. For this purpose, the 50 g of butanol extract are dissolved in 400 ml of mobile phase and added to the separation column. The column is washed with a flow of 1.8 ml/min until 3 l of eluate have dripped. After complete dripping of the remaining mobile phase, the column filling is removed and stirred thoroughly (extracted) in 3 l of 100% methanol for 10 minutes. The stationary phase is filtered off and the eluate is dried. The residue is about 0.5 to 1% of the amount of plant parts used=methanol extract.

### First Fine Separation:

About 4 g of methanol extract are chromatographed over a Sephadex LH20 column (3.5 cm internal diameter and 50 cm height of fill=480 ml Sephadex LH20) with 80% by volume of methanol. For this purpose, the 4 g of butanol extract are dissolved in 40 ml of mobile phase and added to the separation column. The column is operated with a flow of 2.4 ml/min and fractions of 10 min duration each (=24 ml of eluate) are collected. The substance sought is present in fractions no. 48-65. The yield in the case of 4 g of methanol extract is about 0.5 to 1 g.

### Second Fine Separation:

Separation column: 250×30 mm
Stationary phase: Reprosil C18 Aqua 10 μm
Mobile phase: Methanol 35% (V/V)
Flow: 1.5 ml/min.
Fraction size: 10 min=15 ml
Substance I is present in fractions 51 to 52.
The combined fractions 41 to 52 are lyophilized.
Yield about 125 mg of substance I from 4 g of methanol extract.
Chromatographic purity about 97% (HPLC).

### EXAMPLE 2

### Structure Elucidation of the Compound According to Formula I

The compound obtained by separation by column chromatography was characterized using the following apparatuses, and the measured values listed below were obtained.

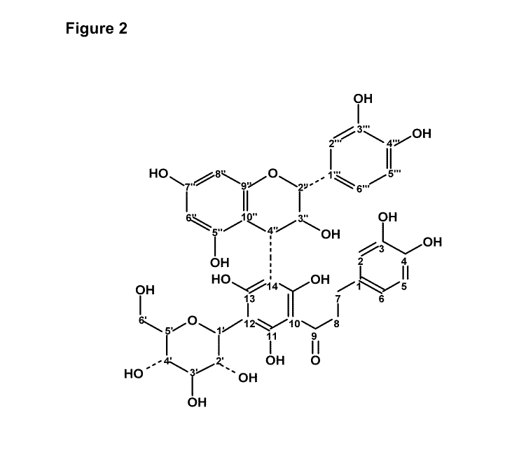

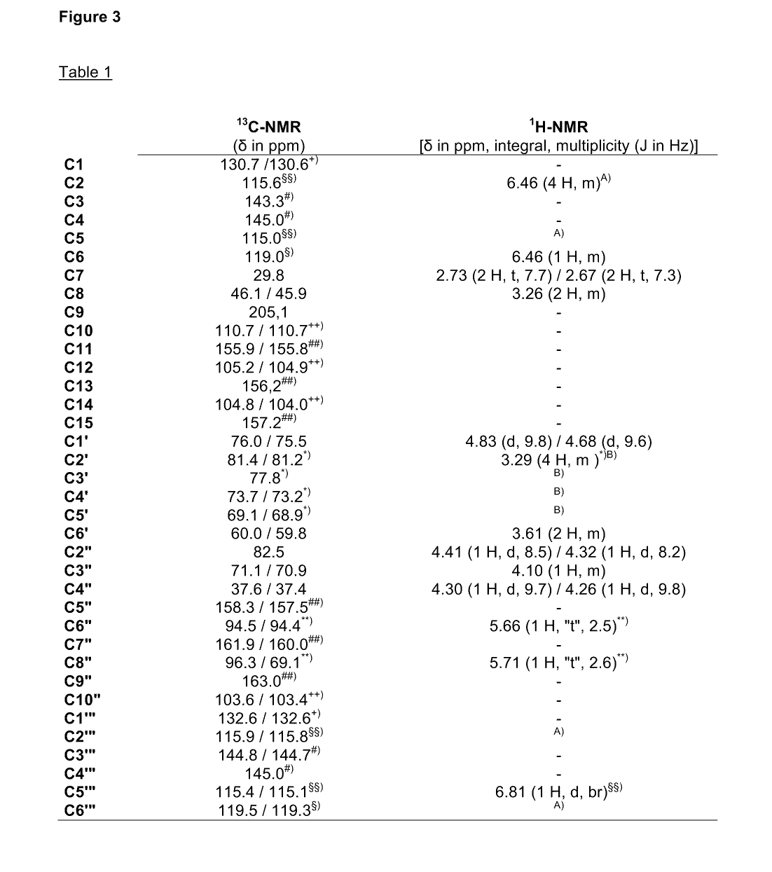

FIG. 2 shows the numbering of the atoms in the inventive compound according to formula I. FIG. 3 shows table 1 with the assignment of the NMR data.

The NMR data were carried out and interpreted as described below.

¹H-NMR (d6-DMSO (Dimethyl Sulphoxide))

The ¹H-NMR of the substance in DMSO gave a spectrum in which nine exchanging phenolic protons and five aliphatic HO protons could be identified by signal reduction (H/D exchange) as a result of addition of deuterated water. In addition, eight protons occur in the olefinic range of 5.5 to 8.0 ppm and nine protons in the shift range typical for —CHₓOH/—CHₓOR groups (x=1, 2). Two aliphatic protons were identifiable in the range around 2.7 ppm, and further aliphatic signals (three protons) overlap with the —CHₓOH/—CHₓOR signals. Some of the signals are doubled or broadened. Individual integrals therefore correspond nominally only to one isomer while others are integrated for both isomers together.

### ¹³C-NMR

The ¹³C-NMR spectrum of the substance shows signal doubling for the majority of the carbon atoms. The ratio of the two isomers varies slightly depending on the solvent used. Eleven signals occur in the range between 25 and 90 ppm for aliphatic carbon atoms and 24 signal groups in the range between 90 and 165 ppm which is typical for olefinic carbon atoms. An additional signal at 205 ppm is overlapped by the solvent but can be unambiguously identified in d6-DMSO. Owing to this overlap, all further 2D experiments were carried out in d6-DMSO.

The measurement of the DEPT (Distortionless Enhancement by Polarization Transfer) spectrum was effected in d₆-acetone. The DEPT spectrum has not only three methylene groups but also an aliphatic methane group and seven-CHOH/—CHOR signals in the range between 65 and 85 ppm. Eight additional signals between 95 and 125 ppm are to be assigned to olefinic CH groups. By matching with the ¹³C-NMR signals, the presence of 17 quaternary carbon atoms can be deduced.

### H/H Correlation (COSY(Correlation Spectroscopy))

The correlation spectrum gives only a few signals which can be evaluated, since a major part of the interactions coincide in the strongly overlapped region of 3.1 to 3.5 ppm. The signal group H2″ to H4″ is clearly identifiable in the range of 4.0 to 4.4 ppm, as is the correlation of the alkyl chain (H7 (2.7 ppm) to H8 (3.3 ppm). Starting from H1′ at 4.7/4.8 ppm, only one correlation is identifiable in the range of 3.1 to 3.5.

C/H correlation spectra (HMQC(Heteronuclear Multiple Quantum Correlation), HMBC(Heteronuclear Multiple Bond Correlation))

The protons neighbouring the quaternary carbon atoms and also the linkage of the individual structural elements can be derived from the CH long-range correlation spectra (HMBC). By assignment of the direct correlations, it is also possible to check whether the structure assignment is plausible.

Evaluation of the Data Obtained and Comparison with Literature Values

The coupling pattern of the protons in the range of 6.8 to 6.4 ppm clearly indicates two greatly similar aromatic systems with 1,3,4-substitution. On the basis of the couplings from the HMBC spectra and the chemical shifts of the carbon atoms involved (145/143/131/119/116/115 ppm), a 3,4-dihydroxybenzylic structure can be derived. A methylene group at 2.7 ppm (30 ppm)—which corresponds to C7—and a —CHOR group at 4.4 ppm (82.5 ppm)—which is assigned to C2″—are identifiable as contact points. The fragment with C7 can be clearly lengthened via the second methylene group (C8: 3.26/46.0 ppm) up to the ketone at C9 (205 ppm). Starting from C9, however, no further correlations are then detectable.

The fragment containing C2″ has a linkage to a further CHOR group (4.1 ppm/71 ppm).

The methane group at 4.3 ppm or 37.5 ppm can be assigned to the signal of C4″ via H/H correlations. From there, further linkage points to two signals in the range of 103 to 105 ppm are detectable, which can be assigned to C14 and C10″, respectively, as well as a CHOR group.

The signal group at 5.7 ppm (2H, 94 or 96 ppm) shows only small meta-couplings (<3 Hz) and can be assigned to a 1,3,5,6-tetrasubstituted aromatic having 3 oxygen functions with further signals at 163/161/158 and 103 ppm. Owing to the different shift, a symmetrical substitution can be ruled out.

The six-CHₓOH/—CHOR groups which are still unassigned cannot be unambiguously assigned since all proton signals and several direct and virtually all long-range couplings coincide in the signal group around 3.3 ppm. From the chemical shifts of the carbon atoms, however, the presence of a carbohydrate side chain can be excluded with good certainty.

The connectivity of the remaining six signals in the aromatic region cannot be unambiguously explained since they are six quaternary signals. These cannot be resolved even via HMBC spectra.

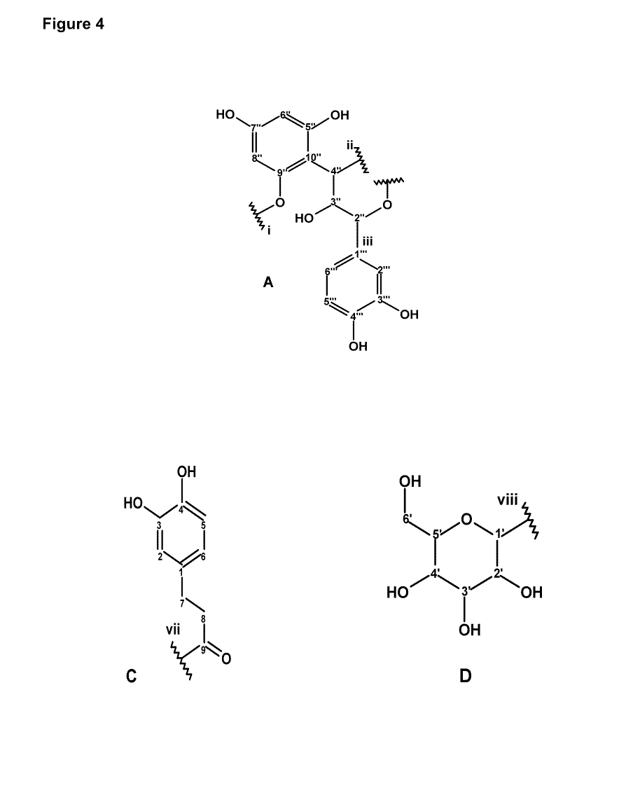

The following structural elements A to D according to FIG. 4 are thus obtained.

On closer consideration, two natural substances can be constructed from these structural elements. Thus, the linkage of i with iii gives a catechin/epicatechin substituted in the C4″ position, while an aspalathin derivative substituted in position C14 is formed by linkage of iv with vii and of v with viii. Both substances are known from rooibos tea. The remaining open linkages C14 and C4″ were already identified as being linked in the course of the assignment of the elements.

A comparison of the ¹³C-NMR data of aspalathin and a C4″-substituted catechin with the compound according to formula I (FIG. 5) gives good agreement of the corresponding values. The presence of conformational isomers (atropisomers, rotational isomers) can be readily explained by the hindrance of the free rotation about the bond C14/C4″.

A comparison with the ¹H-NMR data is dispensed with since, owing to signal superposition and signal doubling, which is due to the presence of rotational isomers, a very complex spectrum is obtained for the compound of the formula I.

Since the substance has not been described in the literature to date, the plausibility of the assignment was checked by means of a simulation program (ADC/C+H NMR Predictor V. 10.02). The agreement was very good on comparison of the ¹³C-NMR data.

The assignment of the three unknown stereocentres and of the carbohydrate is not unambiguously possible; it is effected on the basis of structural analogy and is assigned as a catechinoid all trans-configuration or as a β-gluco configuration.

### Mass Spectrum

Instrument: Micromass Quattro micro API (from Waters); LC-MS
Method: Electrospray ionization (ESI)/secondary ionization EI

### Result

A molecular radical cation at m/z=741 [M+H]⁺ and a cluster ion at m/z=763 [M+Na]⁺ occur in the ESI spectrum. Further fragments occur at m/z=453 [M−catechin]⁺ and 289 [M−catechin−glycoside]. A mass of 740 is to be assigned to the substance.

### UV-Spectrum

The UV spectrum of the substance was recorded in the testing for chromatographic purity by HPLC and diode array detection (DAD) (separation system 1).

### Result

The UV spectrum shows an absorption maximum at 285 nm and a shoulder at 228 nm.

### EXAMPLE 3

### Tele-Stereo-EEG Investigations

The changes in the EEG (electroencephalography) frequencies was determined after administration of saline solution (control) and the compound of the formula I orally (3 or 6 mg/kg body weight).

The investigations were carried out analogously to the method described by W. Dimpfel (Dimpfel, W., Preclinical database of pharmaco-specific rat EEG fingerprints (Tele-Stereo-EEG). Eur. J. Med. Res. (2003) δ: 199-207) as follows: 4 Bipolar concentric electrodes together with a microplug on a common baseplate were implanted in six male adult Fischer-344 rats (converted day-night) aged 6 months. The plug served for recording a four-channel transmitter for telemetric transmission of the field potentials derived from frontal cortex, hippocampus, striatum and reticular formation. The signals were subjected to a fast Fourier transformation on a computer system (“EEG analysis” software, OS science operating system, “LabTeam” laboratory computer from MediSyst, Linden, Germany) in realtime and the power density spectra were determined in each case over 60 minutes. The division of the spectra into 6 different frequency ranges permitted the determination of pharmaco-specific changes in relation to the initial values, measured in each case before application, within these frequency bands.

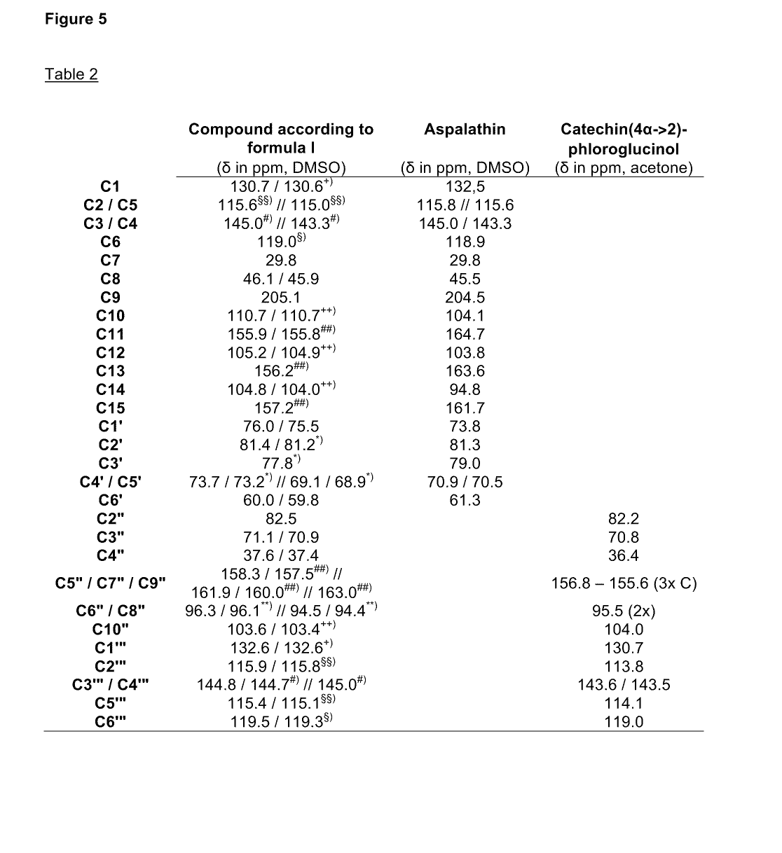

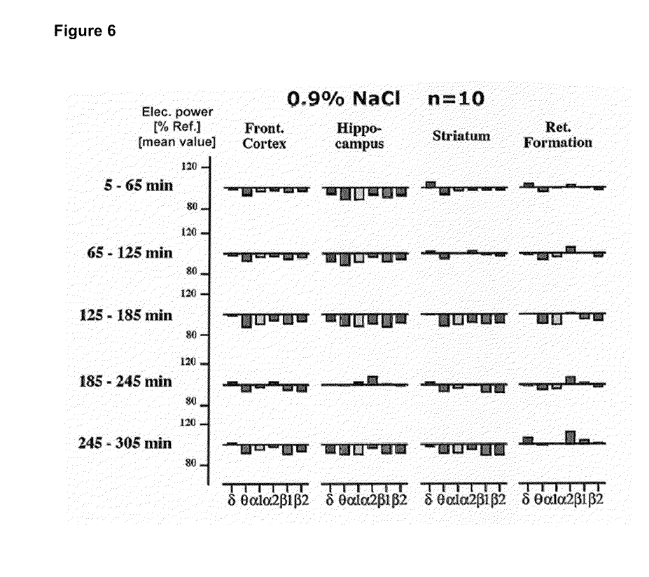

Application protocol of the substances: the substances were applied orally 45 minutes after the beginning of the measurements (initial value). Five minutes later, the measurements were started again, analyzed continuously at least over the next 5 hours and collected in 60 minute periods. The test substance was applied in a dose of 3 and 6 mg/kg (compound of the formula I). The experimental series was begun with the administration of saline solution (control), which led to no abnormal change (FIG. 6).

The statistical comparison of the experiments with the results which were measured after administration of saline solution was effected with the aid of multivariant analysis according to Ahrens and Läuter (cf. H. Ahrens, J. Läuter, “Mehrdimensionale Varianzanalyse” [Multidimensional variance analysis]” (1974), Akademie Verlag, Berlin) on the basis of the changes within the individual frequency bands in all regions of the brain as variables.

The administration of saline solution scarcely led to changes in the electrical activity (pV²/Q) in comparison with the values of the initial phase (FIG. 6).

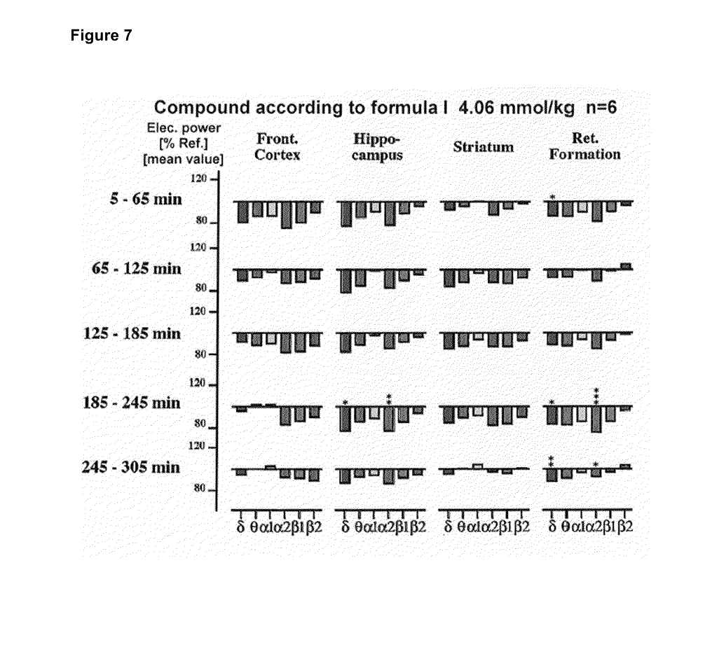

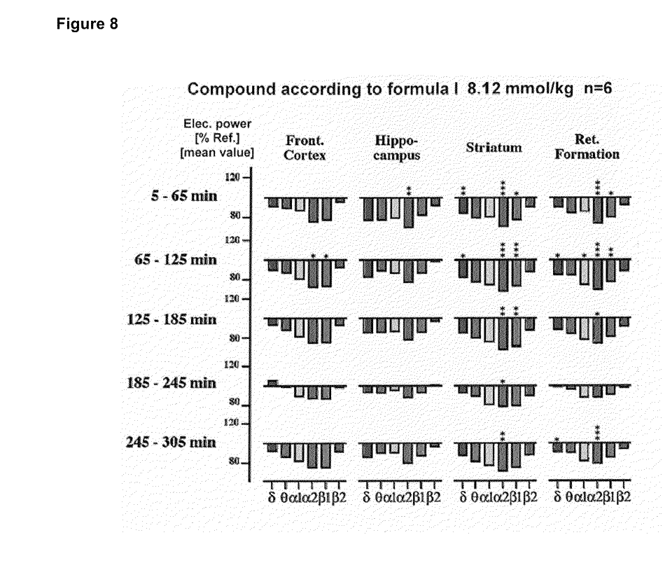

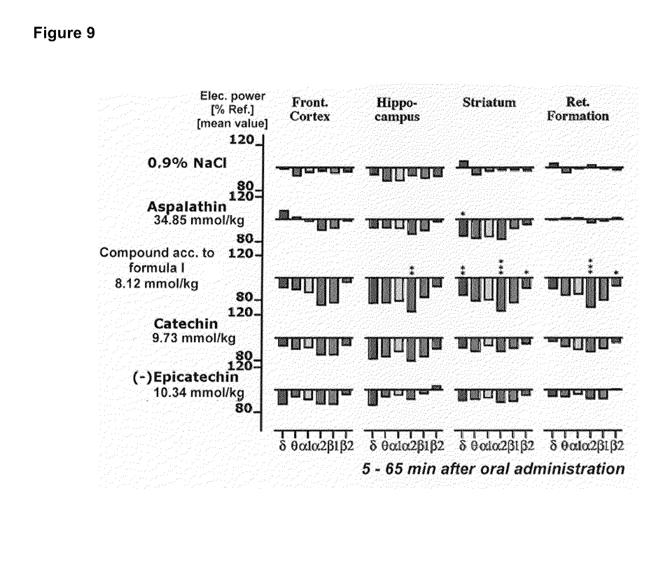

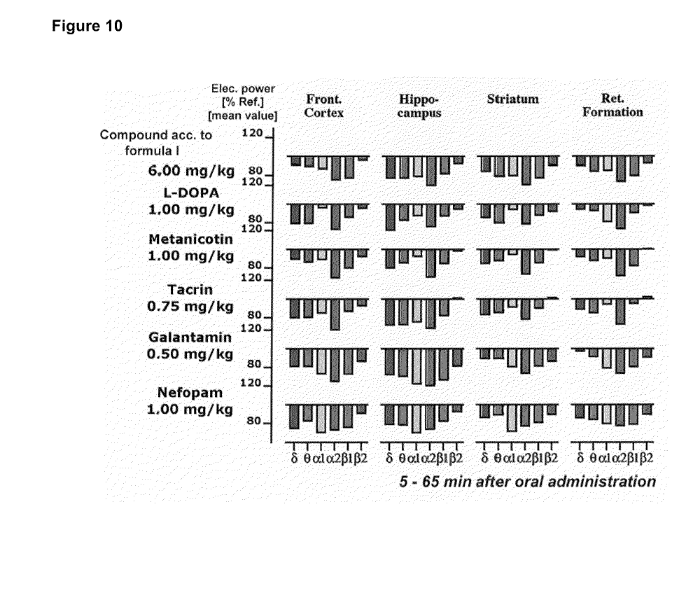

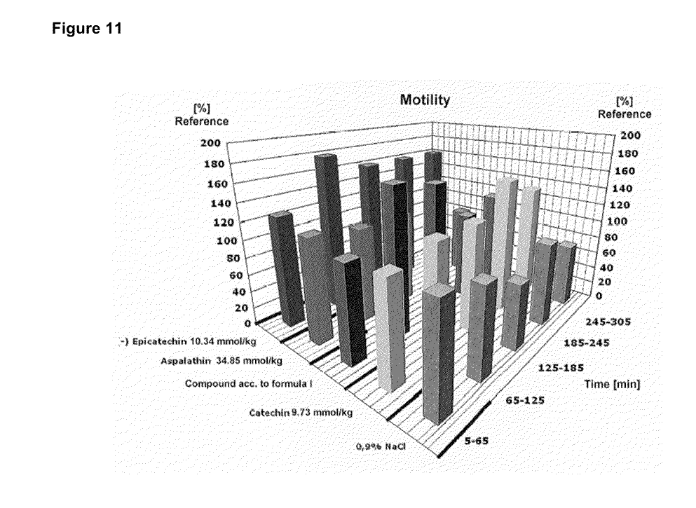

The administration of the compound according to formula I led to stable changes in the power density in all areas of the brain, especially during the second and third hour after application (cf. FIGS. 7 and 8), the changes differing significantly from those after administration of saline solution, in spite of the small number of animals. In the case of approximately equimolar administration of aspalathin, catechin or epicatechin, the effect can be achieved on the basis of the unique structure only by the compound according to formula I but not by parts of the molecule (FIG. 9). This is due to the particular bonding structure of the newly discovered molecule. An increase in the motility was observed only in the presence of (−)-epicatechin (FIG. 11) but not in the presence of the compound according to formula I. This emphasises the independent effect of the compound according to formula I.

The oral administration of the compound according to formula I as a single dose leads to changes in the electrical brain activity in the test animals which corresponded to those after administration of metanicotine, galantamine, tacrine, L-DOPA or nefopam. This pattern correlates in a striking manner with the patterns which occur from medicaments as are usually already used for the treatment of dementia, Parkinson's disease and pain, but not with patterns which occur in the case of medicaments for other indications (FIG. 11).

The observed frequency changes were compared by means of a discriminance analysis with the results of medicaments for the treatment of psychiatric disorders of the brain as well as with medicaments for other indications. The following active substances may be mentioned by way of example: galantamine, nefopam, LSD, metanicotine, caffeine, tacrine, acetylsalicylic acid, methadone, metamizole, fentanyl, fluvoxamine, chlorpromazine, haloperidol, methohexital, meprobamate, midazolam, valproic acid and carbamazepine.

The results of a discriminance analysis based on four regions of the brain and 6 frequency bands (24 variables) showed that the effect of the compound according to formula I is close to that of galantamine, tacrine and nefopam.

### EXAMPLE 4

### Method of Measurement for Determining the Content of the Compound According to Formula I

The content of the compound according to formula I in rooibos and preparations from rooibos (tea, extract, tablets) is determined by means of HPLC/DAD according to the external standard method. For the assay, the substance of the compound according to formula I is used as an external standard. The evaluation is effected at a detection wavelength of 280 nm. In order to prevent oxidation processes in the analysis solution, ascorbic acid is added to the samples.

The preferably used HPLC apparatus is Acquity HPLC/Alliance 2695; detector: DAD, 200 to 400 nm; column: Reprosil-Pur ODS-3, 125×3 mm, 3 μm, from Dr. Maisch; column temperature: 60° C.

Eluent: Eluent A: water/formic acid 100/0.2 (V/V);
Eluent B: acetonitrile/methanol/water/formic acid 50/25/25/0.2 (V/V/V/V).
   Injection volume: 20 μL.
   Running time: 95 min.
   Retention time of the compound according to formula I: 39.5 min.

TABLE 1
(Gradient):

| | Flow rate | | | |
|---|---|---|---|---|
| Time (min) | (ml/min.) | Eluent A % | Eluent B % | Gradient slope |
| 0 | 0.3 | 100 | 0 | |
| 40 | 0.3 | 70 | 30 | linear |
| 60 | 0.5 | 20 | 80 | linear |
| 80 | 0.5 | 20 | 80 | linear |
| 81 | 0.3 | 100 | 0 | |
| 95 | 0.3 | 100 | 0 | |

The following is used as a standard solution:

1 mg of the compound according to formula I, accurately weighed, and about 20 mg of ascorbic acid are dissolved in 2 ml of methanol and made up to 20.00 ml with water.

Required concentration: 0.05 mg/ml of the compound according to formula I and 1 mg/ml of ascorbic acid.

The following is used as an analysis solution:

### Medicament Formulation:

The sample to be investigated, for example a tablet, is pulverized in a powder mill and screened over a sieve having a mesh size of 250 μm.

About 0.5 g of pulverized sample together with about 50 mg of ascorbic acid are accurately weighed into a 50 ml graduated flask, 10 ml of methanol at 40° C. are added and extraction is effected for 10 min in an ultrasonic bath at 40° C. Thereafter, the mixture is made up to the mark with water, vigorously shaken and extracted again for 10 min in an ultrasonic bath at 40° C. After cooling, the mixture is, if required, made up to the mark with water and the solution is centrifuged for 5 min at 9,300 g. The supernatant is filtered over a 0.45 μm membrane filter directly into a small amber glass bottle for the autosampler of the HPLC unit.

### Dry Extract, Extracting Agent Water:

About 125 mg of rooibos extract are weighed together with about 25 mg of ascorbic acid into a 25 ml graduated flask, about 22 ml of water are added and the mixture is vigorously shaken, treated, if required, in an ultrasonic bath and made up to the mark with water.

### Dry Extract, Extracting Agent not Water:

About 125 mg of rooibos extract are weighed together with about 25 mg of ascorbic acid into a 25 ml graduated flask, about 2.5 ml of methanol are added and treatment is effected for 10 min in an ultrasonic bath. Thereafter, the mixture is made up to the mark with water, vigorously shaken and treated again for 10 min in an ultrasonic bath.

### Evaluation:

Standard solution and analysis solution are chromatographed directly in succession under the same conditions. The UV spectra of the reference substance are compared with the substance detected for the same retention time in the analysis chromatogram and, on agreement, the peak is calculated as compound according to formula I according to the following calculation formula:

$$Content [ \% ] = \frac{\begin{matrix}Peak area analysis \cdot dilution of analysis solution ( ml ) \cdot \\ weight taken for standard ( mg ) \cdot 100\end{matrix}}{\begin{matrix}Peak area standard \cdot dilution of standard solution ( ml ) \cdot \\ weight taken for analysis ( mg )\end{matrix}}$$

### EXAMPLE 5A

### Formulations of Monopreparations of the Compound According to Formula I

The expression “compound I” in the examples designates all compounds according to formula I and the pharmaceutically tolerated salts, derivatives and esters thereof.

### Film-Coated Tablet:

| | | |
|---|---|---|
| Compound I | 50 | mg |
| Ascorbic acid | 60 | mg |
| Sorbitol powder (Karion instant) | 120 | mg |
| Aerosil (fumed silica) | 3 | mg |
| Tablet excipient K | 2.8 | mg |
| | 235.8 | mg |

### Film:

| | | |
|---|---|---|
| Shellac | 3.5 | mg |
| Ethanol (solvent for Shellac) | 97%, about 70 | mg |

### Hard Gelatin Capsule:

### Capsule Fill Mass:

| | | |
|---|---|---|
| Compound I | 20 | mg |
| Ascorbic acid | 60 | mg |
| Maltodextrin | 120 | mg |
| Aerosil (fumed silica) | 2 | mg |
| Magnesium stearate | 1.5 | mg |

### EXAMPLE 5B

### Formulations of Monopreparations with Extract Containing the Compound According to Formula I

### Film-Coated Tablet:

| | | |
|---|---|---|
| Extract (with compound according to formula I) (1%) | 150 | mg |
| Ascorbic acid | 60 | mg |
| Sorbitol powder (Karion instant) | 80 | mg |
| Aerosil (fumed silica) | 3 | mg |
| Magnesium stearate | 2.5 | mg |
| | 295.5 | mg |

### Film:

| | | |
|---|---|---|
| Shellac | 4.2 | mg |
| Ethanol (solvent for shellac) | 97% about 84 | mg |

### Hard Gelatin Capsule:

### Capsule Fill Mass:

| | | |
|---|---|---|
| Extract (with compound according to formula I)(1%) | 150 | mg |
| Ascorbic acid | 40 | mg |
| Maltodextrin | 20 | mg |
| Aerosil (fumed silica) | 1 | mg |
| Magnesium stearate | 1.5 | mg |
| | 212.5 | mg |

### EXAMPLE 6

### Extract with Increased Content of Substance of the Formula I and Increased Total Flavonoid Content

### a) Extraction

Leaves and sprigs of Aspalathus linearis (unfermented) dried particularly rapidly and under mild conditions—“green” rooibos having as low a residual moisture content as possible (<5%)—were used as the starting drug.

The extract was obtained by various extracting agents: 20% methanol or 30% ethanol and 50% ethanol in water. Alternatively, the drug can first be added to the pure alcohol and, after a softening phase of at least 30 minutes, the corresponding amount of water can be added.

These extractions are optimized for the complete as possible extraction of compounds of the formula I and at the same time the total flavonoids.

The extract differs from the commercially available extracts to date for internal use in tea beverages (purely aqueous extraction) through higher contents of total flavonoids; it differs from the extract for external use in cosmetics (80% ethanol) through the ratio of the flavonoid groups to one another. The 80% ethanol extract was optimized for as high an aspalathin content as possible. The ratio of the 3 flavonoid groups (aspalathin type, rutoside type and vitexin type ═C-glycosides) is changed by the relatively lipophilic extraction compared with the initial state in the drug. This is particularly clearly recognizable from the ratio of the vitexin-like flavonoids to the rutoside-like flavonoids.

b) Method of Measurement for Analytical Determination of the Components from the Crude Extracts

The ascorbic acid and the solvents used were obtained from Roth (Karlsruhe, Germany) and were of analytical quality. The drug was obtained from Rooibos Ltd., Clan William, South Africa.

The standards orientin, homoorientin and vitexin are commercially available, for example from Extrasynthese (Genay, France) or from Roth (Karlsruhe, Germany).

The syringe filter used was a 13 mm Rotilabo syringe filter (0.45 μm, PVDF) from Roth (Karlsruhe, Germany).

For the HPLC measurement, the HP 190 Series II Liquid Chromatograph apparatus with diode array Detector E-3014 (Hewlett Packard) with the ChemStation software for LC 3D Rev. A.10.02 (Agilent Technologies) and the Reprosil-Pur ODS-3 column, 125×3 mm, 3 μm (from Dr. Maisch, Ammerbuch, Germany) was used at a column temperature of 60° C.

The mobile phase A: water/formic acid 100/0.2 (V/V) and mobile phase B: acetonitrile/methanol/water/formic acid 50/25/25/0.2 (V/V) were used, the following conditions being chosen:

### Gradient:

TABLE 2
Gradient:

| | Flow rate | | | |
|---|---|---|---|---|
| Time [min] | [ml/min] | MP A [%] | MP B [%] | Gradient |
| 0 | 0.3 | 100 | 0 | |
| 40 | 0.3 | 70 | 30 | Linear |
| 60 | 0.5 | 20 | 80 | Linear |
| 80 | 0.5 | 20 | 80 | Linear |
| 81 | 0.3 | 100 | 0 | |
| 95 | 0.3 | 100 | 0 | |

Injection volume: 20 μl; running time: 95 min

1.0 mg of standard substance, accurately weighed, and about 20 mg of ascorbic acid are dissolved into 2.0 ml of methanol and made up to 20.0 ml with water. The following standards were used: homoorientin, orientin, vitexin, rutoside, isoquercitrin, ferulic acid.

As a standard solution of compounds of formula I, a solution of the extract G110907SA in methanol was used as a working standard. For this purpose 125 mg of extract G110907SA together with about 25 mg of ascorbic acid were weighed into a 25 ml graduated flask and about 22 ml of water were added. After vigorous shaking and treatment in an ultrasonic bath, the mixture is made up to the mark with water. The content of compounds of the formula I in this extract is 0.95% and was used for the assay in the other extracts.

### c) Measurements

6.c)1) Extraction: 20% Methanol Extracts

0.5 g of extract (accurately weighed) is weighed together with about 50 mg of ascorbic acid into a 50.0 ml graduated flask, 10 ml of methanol (40° C.) are added and extraction is effected for 10 min in an ultrasonic bath at 40° C. The mixture is then made up to the mark with water, vigorously shaken and extracted again for 10 min in an ultrasonic bath at 40° C. After cooling, the solution is, if required, made up to the mark with water and centrifuged for 5 min at 9300×g.

1 ml of the supernatant was taken, filtered over a syringe filter into a vial (amber glass) and measured by means of HPLC.

6.c)₂) Compound of the Formula I

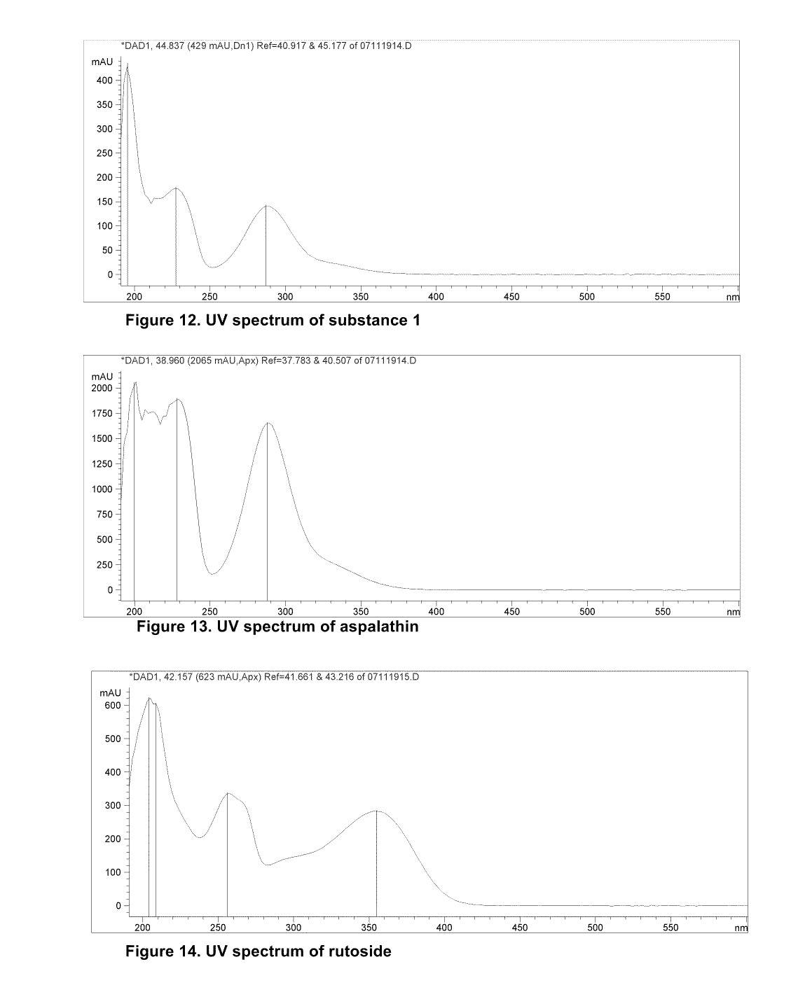

The identification of the substance 1 in the chromatograph of the extract 1 (G110907SA) was effected via an available comparison spectrum and via the UV comparison spectrum (FIG. 12) and an online UV spectrum.

The content of substance 1 in various extracts was calculated using the known content in the extract 1 (G110907SA) (0.95%) according to the following formula (A-area, V-volume, m-mass, g-content, Ana-analysis, St-standard):

$${g}_{Substance 1} ( \% ) = \frac{{A}_{Ana} {V}_{Ana}}{{m}_{Ana} {relA}_{St}} 100 \%$$

the relative area relA being calculated as follows:

$$relA = \frac{100 {A}_{St}}{{V}_{Extract} {m}_{Extract} 0.95} = 12.52 \pm 0.04$$

6.c)₃) Flavonoids of the Substance A Group

Flavonoids of the substance A group are characterized by UV maxima at 287 nm and 228 nm. All peaks which show substantial agreement with this spectrum (FIG. 13) are included in this group and the content calculated according to the following formula:

$${g}_{FlavonoidgroupA} ( \% ) = \frac{{A}_{Ana - GroupA} {V}_{Ana} {m}_{Rutosid}}{{m}_{Ana} {A}_{Rutoside} {V}_{Rutoside}} {k}_{f} 100 \%$$

the standard used is rutoside and the correction factor k_{f} is 0.4.
6.c)₄) Flavonoids of the Rutoside Group

Flavonoids are assigned to this group on the basis of a rutoside comparison spectrum (FIG. 14). The content is calculated according to the following formula:

$${g}_{FlavonoidRutoside} ( \% ) = \frac{{A}_{Ana - Rutoside} {V}_{Ana} {m}_{Rutoside}}{{m}_{Ava} {A}_{Rutoside} {V}_{Rutoside}} 100 \%$$

6.c)₅) Flavonoids of the Vitexin Group

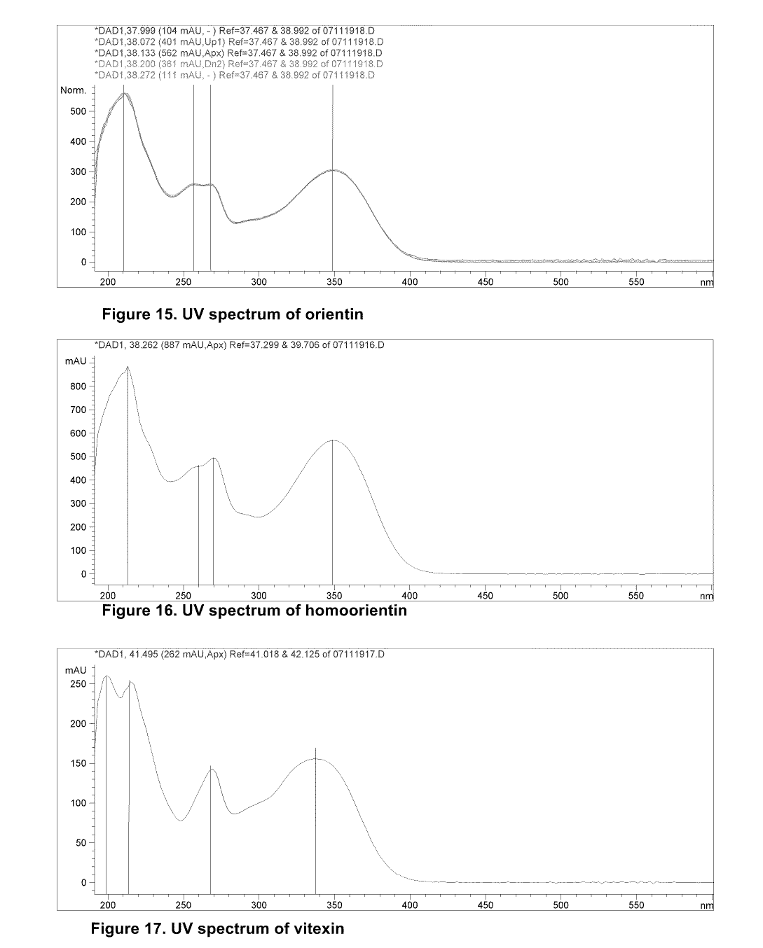

Flavonoids are assigned to this group on the basis of a vitexin comparison spectrum to which orientin and homoorientin also belong (FIG. 15 to FIG. 17). Instead of vitexin, homoorientin is used as a standard since vitexin (and also orientin) showed problems with the solubility in the preparation of the standard solutions. The content is calculated according to the following formula:

$${g}_{FlavonoideVitexin} ( \% ) = \frac{{F}_{Ana - Vitexin} {V}_{Ana} {m}_{Homoorientin}}{{m}_{Ana} {F}_{Homoorientin} {V}_{Homoorientin}} 100 \%$$

### 6d) Results

The results obtained in the analysis are summarized in table 3 below:

In the case of the extract according to the invention, efforts were made to obtain the 3 flavonoid groups in the extract as far as possible in the same ratio as they are present in the starting drug (taking into account the natural variation in the drug and due to the process from batch to batch). This ratio is then comparable with the ratio in the customary tea beverages from green rooibos of high quality (lower degree of fermentation).

TABLE 3
Content of flavonoids - substance 1, rutoside group, substance A
and vitexin group, based on the drug used and based on the
differently prepared extracts (starting from the drug CH
G310807SA). The % contents are in each case mean values
of at least 2 extract work-ups. (A) The extraction was first
carried out for 10 minutes with the pure alcohol and water was
then added until the stated alcohol content was established.
(B) The extraction was carried out from beginning to end
with an alcohol/water mixture having the stated alcohol content.

| | | Content | | | |
|---|---|---|---|---|---|
| | | [%] | | | Drug/ |
| | Substance | Rutoside | Substance | Vitexin | Extract |
| | I | Group | A Group | Group | Ratio |
| Drug | 1.09 | 0.56 | 5.81 | 0.87 | |
| (G310807SA) | | | | | |
| Extract(A): | 2.12 | 1.85 | 13.53 | 2.52 | 3.2:1 |
| 20% Methanol | (95% | (230% | 133% | (190% | |
| | 1.9 times) | 3.3 times) | 2.3 times) | 2.9 times) | |
| Extract(A): | 2.66 | 2.03 | 13.51 | 2.14 | 3.2:1 |
| 30% Ethanol | (144% | (262% | (132% | (145% | |
| | 2.4 times) | 3.6 times) | 2.3 times) | 2.5 times) | |
| Extract(B): | 2.57 | 2.45 | 15.65 | 3.14 | 3.4:1 |
| 20% Methanol | (136% | (338% | (169% | (261% | |
| | 2.4 times) | 4.4 times) | 2.7 times) | 3.6 times) | |
| Extract(B): | 3.02 | 2.26 | 14.51 | 2.85 | 3.2:1 |
| 30% Ethanol | (177% | (304% | (149.7% | (227% | |
| | 2.8 times) | 4.0 times) | 2.5 times) | 3.3 times) | |

In each case the percentage by which, or the number of times by which, the corresponding flavonoid is present in the respective extract in comparison with the drug is inserted in brackets.

### EXAMPLE 7

### Further Purification

An extract having an even higher content of substance of the formula I and total flavonoids was prepared.

The extract was first prepared as described in example 6a) and then further purified by purification over a Sephadex column similarly to the 1st Sephadex step for obtaining the pure substance according to example 1.

Content of compound of the formula I: >5%.
Total flavonoid content (sum of aspalathin type+rutoside type+vitexin type without
compound according to formula I: >35%
Ratio of vitexin type (═C-glycosides) to rutoside type: </=1.6
Total aspalathin type: >25%.

The approximate retention times (HPLC, cf. example 6, Determination of the contents) were as follows under the stated conditions:

Compound according to formula I: 45 min.

### Aspalathin: 39 min.

### Rutoside: 42 min.

### Orientin/Homoorientin: 38 min.

Vitexin: 41.5 min.

1. A compound of the formula I 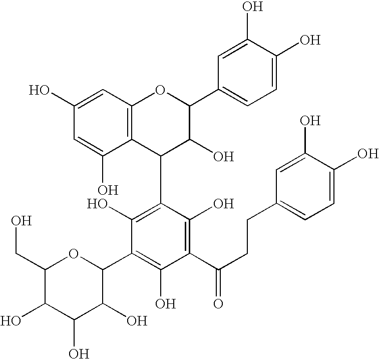
or a pharmaceutically acceptable salts, derivative or esters thereof.

2. A rooibos extract containing the compound of the formula I according to claim 1, characterized in that the rooibos extract is obtained from unfermented rooibos.

3. The rooibos extract according to claim 2, characterized in that said extract contains at least 1.0% by weight of the compound according to formula I.

4. The rooibos extract according to claim 3, characterized in that said extract contains at least 2.0% by weight of compound of the formula I.

5. The rooibos extract according to claim 2, characterized in that the total flavonoid content is at least 20% by weight.

6. A process for the preparation of the compound of the formula I according to claim 1 and a rooibos extract obtained from unfermented rooibos containing the compound of the formula I according to claim 1, said process comprising the steps of:
extracting dried and comminuted, unfermented raw rooibos material with an extracting agent consisting of 10 to 60% (vol/vol) alcohol with water as the remainder for a predetermined duration of extraction at a temperature of up to 90° C.,
filtering the extract and then evaporating it to dryness under reduced pressure.

7. The process according to claim 6, further comprising at least one chromatographic separation step to effect more substantial purification.

8. The process according to claim 7, wherein the chromatographic separation step is size exclusion chromatography.

9. A food supplement containing at least 1 mg of the compound of the formula I according to claim 1 per g of food supplement.

10. A food supplement containing the rooibos extract according to claim 2 in an amount of at least 10 mg/g of food supplement.

11. The food supplement according to claim 9, wherein said food supplement is formulated in such a wav that the daily dose thereof contains at least 1 mg of the compound of the formula I according to claim 1.

12. A medicament containing the compound of the formula I according to claim 1 or a rooibos extract obtained from unfermented rooibos containing the compound of the formula according to claim 1.

13. A method for preventing and/or treating neurological and psychiatric disorders of the central nervous system comprising the step of administering a compound of formula I according to claim 1 or a rooibos extract obtained from unfermented rooibos containing the compound of the formula I according to claim 1.

14. The method according to claim 13, wherein the neurological and psychiatric disorders of the central nervous system are dementias, Parkinson's disease, depression and pain.

15. The method according to claim 14, wherein the disorder of the central nervous system is Alzheimer's disease or senile dementia.

16. The process according to claim 6, wherein the extraction step is performed at a temperature of up to 60° C.

17. The food supplement according to claim 9, wherein said food supplement is formulated in such a way that the daily dose thereof contains at least 5 mg of the compound of the formula I according to claim 1.
